(19) United States
(12) Patent Application Publication
(10) Pub. No.: US 2005/0020910 A1
(43) Pub. Date: Jan. 27, 2005

(54) Intra-oral imaging system

(76) Inventors: Henley Quadling, Addison, TX (US); Mark Quadling, Plano, TX (US)

Correspondence Address: BRINKS HOFER GILSON & LIONE, P.O. BOX 10395, CHICAGO, IL, 60610, US

(21) Appl. No.: 10/837,089

(22) Filed: Apr. 30, 2004

Related U.S. Application Data
(60) Provisional application No. 60/466,549, filed on Apr. 30, 2003.

(51) Int. Cl. A61B 6/00; A61B 5/05

(52) U.S. Cl. 600/424; 600/476

(57) ABSTRACT

A digitized image of a tangible object is displayed in an operator's field of view of the object almost simultaneously as the digitized image is being captured. The image is projected onto a screen in an orientation, position and scale corresponding to an orientation and position of the object within the field of view of the operator so as to be perceived as an overlay to the object. The image may be a one-, two, three, or other multi-dimensional representation of the object and may be captured by an imaging system, such as an intra-oral imaging device.

### PRIORITY AND CROSS-REFERENCE TO RELATED APPLICATIONS

This application claims the benefit under 35 U.S.C. § 119(e) of co-pending provisional application No. 60/466,549 filed on Apr. 30, 2003, for Digitizing/Imaging System with Head-Mounted Display For Dental Applications, which is incorporated in its entirety herein by reference.

### BACKGROUND OF THE INVENTION

1. Related Field

The invention relates to three-dimensional imaging of objects. In particular, the invention relates to displaying a three-dimensional image of an intra-oral (in vivo) dental item that may include dentition, prepared dentition, restorations, impression materials and the like.

2. Description of the Related Art

Existing intra-oral imaging systems may use a Moiré imaging technique. With Moiré imaging, a three-dimensional (“3D”) image of a physical object may be generated by scanning the object with white light. The 3D image may be viewed on a display or video monitor. Operators may evaluate the 3D image only through the display, which may require the operator to look away from the object. In addition, there may be little or no feedback as to whether the image is suitable for its intended purpose.

### SUMMARY OF THE INVENTION

An imaging embodiment projects or displays a computer-generated visual image in a field of view of an operator. The systems, methods, apparatuses, and techniques digitize physical objects, such as dental items. The image may be displayed on and viewed through a head-mounted display (“HMD”), which displays computer-generated images that are easily viewed by the operator. The image also may be displayed on a computer monitor, screen, display, or the like.

A computer-generated image may correspond to an image of a real-world object. The image may be captured with an imaging device, such as an intra-oral imaging system. The intra-oral imaging embodiment projects structured light toward tissue in an oral cavity so that the light is reflected from a surface of that tissue. The tissue may include a tooth, multiple teeth, a preparation, a restoration or other dentition. The intra-oral imaging embodiment detects the reflected white light and generates a dataset related to characteristics of the tissue. The dataset is then processed by a controller to generate a visual image. The controller-generated visual image may be displayed on a screen in the HMD. The image may be displayed at a position and/or orientation corresponding to position and/or orientation of the tissue within the field of view of an operator. The imaging embodiment senses changes in a field of view of an operator, such as by movement of the operator's head, and adjusts the position and/or orientation of the image to correspond with the changes in the field of view of the operator.

An exemplary intra-oral imaging system includes an imaging device, a processor and a head mounted display. The imaging device may project light towards or onto a surface of the object so that the light is reflected from the object. The imaging system generates a dataset that represents some or substantially of the surface characteristics of the object. The imaging system may include a tracking sensor that tracks a position of the imaging system relative to the head-mounted display. The tracking sensor may detect an orientation of the imaging system to provide temporal orientation information. The tracking sensor also may detect a position of the imaging device to provide temporal position information. The orientation information may include data related to various angles of the imaging device relative to a predetermined origin in free space. The position information may include data related to a distance or position measurement of the imaging device relative to a predetermined origin in free space. The orientation information may include data for multiple angles, such as three angles, and the position may include measurements along multiple axes, such as three axes. Accordingly, the tracking sensor may provide information for multiple degrees of freedom such as the six-degrees of freedom described above. The dataset generated by the imaging system may also correspond to a two-dimensional or a three dimensional representation of the surfaces of an object.

The imaging device may manipulate the properties of white light through Moiré or image encoding, laser triangulation, confocal or coherence tomography, or wave front sensing. The coherence tomography imaging may digitize a surface representation of the object that may be visually occluded. For example, an imaging device based on coherence tomography may capture an image of the tooth structure behind soft tissues such as the underlying gum tissue, other soft matter such as tartar, food particles, or any other material.

A processor may receive the dataset from the imaging device. Based on the information contained in the dataset, the processor may generate signals representative of a visual image of the surface of the object. The processor may generate signals substantially simultaneously as the generation of the dataset by the imaging system. The processor also may generate signals in response to receiving the dataset or as the dataset is received. The processor may be coupled to the imaging system through a link that may include wires, cables, via radio frequency, infra-red, microwave communications and/or some other technology that does not require physical connection between the processor and imaging system. The processor may be portable and may be worn by the operator.

The HMD may be fitted or otherwise coupled to the head of an operator. The HMD receives the signals from the processor. Based on the signals received from the processor, the HMD may project the image onto a screen positioned in the field of view of an operator. The HMD may project the image to be seen by one or both eyes of the operator. The HMD may project a single image or a stereoscopic image.

The HMD may include a HMD position sensor. The position sensor may track the HMD's position relative to a predetermined origin or reference point. The position sensor also may detect an orientation of the HMD to provide HMD orientation information as a function of time. The position sensor may also detect a position of the HMD to provide position information of the HMD as a function of time. The orientation information may include data related to various angles of the HMD relative to the predetermined origin. The position information may include data related to a distance or position measurement of the HMD relative to the predetermined origin. The orientation information may include data for one or more angles and the position may include measurements along one or more axes. Accordingly, the sensor may provide information for at least one or more degrees of freedom. The HMD position sensor may include optical tracking, acoustic tracking, inertial tracking, accelerometer tracking, magnetic field-based tracking and measurement or any combination thereof.

The HMD also may include one or more eye tracking sensors that track limbus or pupil, with video images or infrared emitters and transmitters. The location and/or orientation and the location of the operator's pupil are transmitted at frequent intervals to a processing system such as a computer coupled to an intra-oral probe.

The intra-oral probe may include a multi-dimensional tracking device such as a 3D tracking device. A 3D location of the probe may be transmitted to a controller to track the orientation and location of the probe. A 3D visualization of an image of the object may be displayed to the operator so that the operator can view the image over at least a portion of the actual object being digitized. The operator may progressively digitize portions of the surface of the object including various surface patches. Each portion or patch may be captured in a sufficiently brief time period to eliminate, or substantially reduce, effects of relative motion between the intra-oral probe and the object.

Overlapping data between patches and a 3D localization relationship between patches may be determined based on the localization information received from the tracking sensor and the HMD position sensor. In addition, overlap between the digitized image of the object and the operator's eye may also be determined. Simultaneous, or substantially instant, feedback of the 3D image may be transmitted to the HMD to allow the image to be displayed in real-time. The computer-generated image may be displayed localized in the operator's field of view in about the same location as the actual object being digitized. The generated image also may be displayed with a scaling and orientation factors corresponding to the actual object being digitized. Gaps in the imaged surface, as well as crucial features may be enhanced to alert the operator to potential issues. Triangulation shadowing and other issues may be communicated to the operator in a visual and/or intuitive way. The intra-oral imaging system may provide substantially instant and direct feedback to an operator regarding the object being imaged.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 illustrates an example of the intra-oral digitizing embodiment.

FIG. 2 illustrates an operator wearing a head mounted display.

FIG. 3 illustrates a side view of the operator wearing the head mounted display.

### DETAILED DESCRIPTION OF THE INVENTION

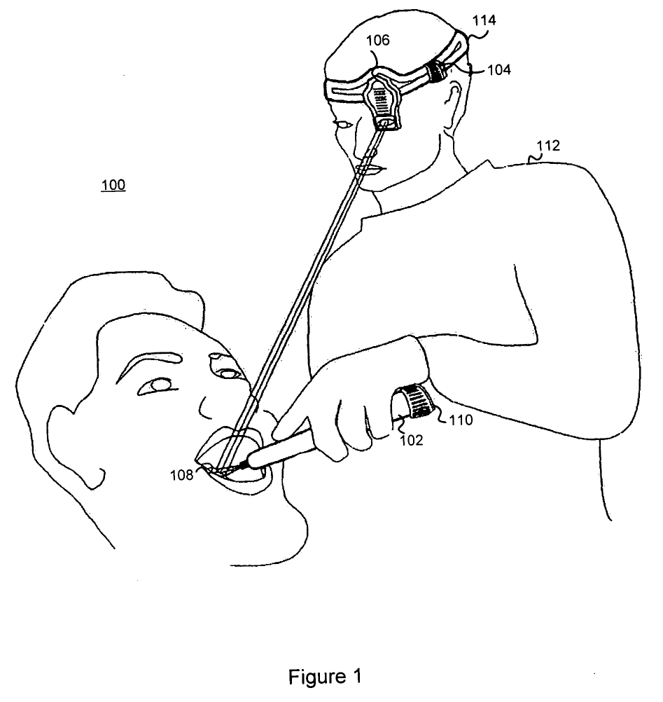

FIG. 1 illustrates an exemplary intra-oral imaging system 100 having an imaging device 102, a processor 104, and a head mounted display (HMD) 106. The HMD may be worn by an operator 112 of the intra-oral imaging system 100. The intra-oral imaging system 100 displays a computer-generated image in the HMD 106. The computer-generated image may illustrate a tangible object 108 in an operator's view. The object 108 may be intra-oral tissue, such as all or portions of a tooth, multiple teeth, a preparation, a restoration, or any other dentition or combination. The computer-generated image may be projected in the field of view of the operator 112.

The imaging device 102 may capture an image of the object 108. The imaging device 102 may be an intra-oral imaging device, such as the Laser Digitizer System For Dental Application disclosed in co-owned application Ser. No. ______, referenced by attorney docket number 12075/37, filed on Mar. 19, 2004, the disclosure of which is incorporated by reference in its entirety. The imaging device 102 also may be an intra-oral imaging device, such as the Laser Digitizer System For Dental Application disclosed in co-owned application Ser. No. 10/749,579, filed on Dec. 30, 2003, the disclosure of which is also incorporated by reference in its entirety. The imaging device 102 projects structured light towards the object 108 so that the light is reflected therefrom. The imaging device 102 scans a surface of the object with the structured light so that the reflected structured light may be detected. The imaging device 102 detects the reflected light from the object 108. Based on the detected light, the imaging device 102 generates a dataset related to surface characteristics of an object. The imaging device may include a processor and memory devices that generates a dataset. The dataset may relate to a two-dimensional image of the object 108, the scanned surface of the object, or one or more portions thereof. The dataset also may relate to a three-dimensional image of the object, a scanned surface of the object, or one or more portions thereof.

The imaging device 102 may generate the dataset based on many white light projection techniques, such as Moiré or laser triangulation. The imaging device 102 may generate the dataset based on image encoding such as light intensity or wavelength encoding. The imaging device 102 also may generate the data set based on laser triangulation, confocal or coherence tomography, wave front sensing or any other technique.

In an embodiment based on coherence tomography, the dataset generated by the imaging device 102 include data related to a surface of the object 108 that may be visually blinded behind other surfaces or materials. For example, the imaging device 102 based on coherence tomography may generate a dataset that includes information related to a surface of the tooth structure behind soft tissues such as the underlying gum tissue, or other soft matter such as tartar, food particles, and/or any other materials.

The imaging device 102 may include a tracking sensor 110. The tracking sensor 110 senses the position of the imaging device. The tracking sensor 110 senses the position of the imaging system in free-space, for example in three degrees of freedom. The tracking sensor 110 may be a magnetic field sensor, an acoustical tracking sensor, an optical tracking sensor such as a photogrammetry sensor, an active IR marker, or a passive IR marker or any other tracking sensor. The tracking sensor 110 may include one or more sensors positioned on the imaging device 102. An example of a tracking sensor 110 includes the Liberty Electromagnetic tracking system, by Polhemus of Colchester, Vt., which may produce a data stream of at least 100 updates per second, where each update includes information concerning the location in a multi-dimensional space of each of a number of sensors placed on the imaging device 102. By tracking the position coordinates of each of the sensors placed on the imaging device 102, the imaging device 102 may be sensed in six degrees of freedom. The six degrees of freedom may specify the position and orientation of the imaging device 102 for each update period.

A processor 104 may be coupled to the imaging device 102. The processor 104 may be a component of or a unitary part of the imaging device 102. The processor 104 and the imaging device 102 may be coupled through a data link including wires, cables, radio frequency, infra-red, microwave communications or other wireless links. The processor may also include communications device that provide for wireless communication protocol, such as wireless TCP/IP for transmission of bidirectional data. The processor 104 may be portable and may be worn around any portion of an operator or carried the operator.

The processor 104 may receive datasets from the imaging device 102. Based on the dataset, the processor 104 may generate image signals. The image signal may be characterized as a digital or logic signal, or an analog signal. The processor 104 generates the image signal based on the captured images from the imaging device 102. The image signal represents a computer-generated image, or visual representation, of a captured image of the object 108, an image if the surface of the object 108 or a portion thereof.

In one embodiment, the processor 104 may generate the image signal in response to, and substantially simultaneously with, the generation of the dataset by the imaging system 102. The processor 104 also may generate the image signals when receiving the dataset.

The processor 104 also may receive tracking information from a tracking sensor 110. Based on the information received from the tracking sensor 110, the processor 104 may align or calibrate a projected image of the object with a captured image of the object 108. The processor 104 may includes a wireless transmitter and antenna 28 for wireless connectivity to an open or private network or to a remote computer or terminal.

The HMD 106 is coupled to the processor 104 to receive the image signal generated by the processor 104. The HMD 106 may be coupled to the processor through a data link including wires, cables, radio frequency, infra-red, microwave communications or other wireless links. The processor 104 also may be a unitary part of the HMD 106.

The HMD 106 receives the image signals from the processor 104. Based on the image signals, the HMD 106 may display a controller-generated image to the operator 112. The HMD 102 may use an image display system positioned in the line of sight of the operator 112. Alternatively, the display system may project the controller-generated image in a field of view of the operator 112. An example of such an image display is the Nomad display sold by Microvision Inc, of Bothell Wash. The image may include detailed information about the image capture process, including a visualization of the object 108 or portion thereof. The information also may include analysis of the dataset.

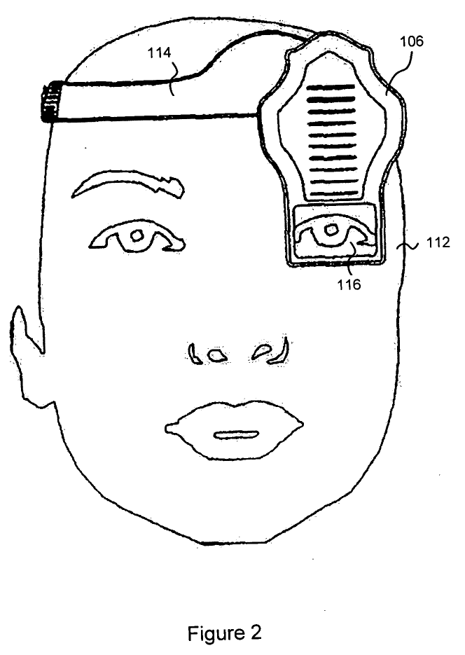

FIG. 2 illustrates an example of the HMD 106 worn by an operator 112. The HMD 106 includes a screen 116 that may display the controller-generated image. The screen 116 may include transparent, or semi-transparent, material that reflects or directs the controller-generated image towards the operator 112. The HMD 106 may be positioned so that the operator 112 can view images displayed on the screen 116. The image may be projected on the screen 116 in the field of view of the operator 112. The image may be projected on the screen 116 in a position and orientation that overlays the object 108 within the field of view of the operator 112. By projecting the image onto the screen 116, the operator's view may be augmented or enhanced. The image may also include graphics, data, and textual information.

In a second embodiment, a headband 114 is used to position the HMD 106 on the operator's head so that the screen 116 is in the field of view of the operator 112. The screen may be positioned in front of, or before, at least one of the operator's eyes. The processor 104 also may be affixed to the headband 114. In one embodiment with the processor 104 coupled to the headband 114, the headband 114 may provide a channel for routing wires between the processor 114 and the HMD 106.

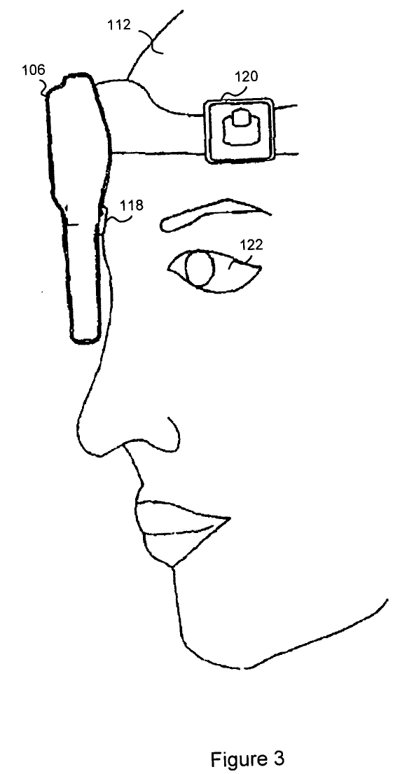

In a third embodiment, the intra-oral imaging system 100 includes an eye tracking sensor 118. FIG. 3 illustrates a side view of the HMD 106 worn by an operator 112 having an eye tracking sensor 118. The eye tracking sensor 118 may be affixed to the HMD 106. The eye tracking sensor 118 may be coupled to or a unitary part of the HMD 106.

The eye tracking sensor 118 may track or detect movement, location, orientation of the operator's eye 122. By tracking the operator's eye 122, the eye tracking sensor may provide feedback on the operator's line of vision. The eye tracking sensor 118 may also detect the operator's line of vision with respect to an object 108 or with respect to the operator's environment. The eye tracking sensor 118 provides a signal to the processor 104 corresponding to operator's line of sight. The processor 104 receive the signal from the eye tracking sensor 118 and may store the position and view of the eye 112 to the image displayed on the screen 116. The processor may also store the position and view of the eye 112 relative to the actual scene. Alternatively, the eye tracking sensor 118 may register the operator's line of sight with respect to the screen 116.

The eye tracking sensor 118 may track various areas of the eye 122 such as the limbus, the cornea, retina, pupil, sclera, fovea, lens, iris, or other parts of the eye 122. In one embodiment, the eye tracking sensor 118 employs a video camera to track the eye 122. In another embodiment, the eye tracking sensor may use infrared emitters and transmitters to track the eye 122. Location and orientation parameters are provided to the processor 104 at predetermined frequent intervals to provide substantially real-time feedback to the processor 104. An example of an eye and head tracking system that measures eye movement substantially in real-time and point-of-regard data is the VisionTrak head mounted eye tracking system sold by Polhemus of Colchester Vt.

The HMD also may include one or more position sensors 120. The position sensor 120 may provide a position signal to the processor 104 related to the position, location and orientation of the operators head. The position sensor 120 may produce position information in multiple degrees of freedom. The signal provided by the position sensor 120 allows accurate alignment of the projected and captured images. The position sensor may be a magnetic field tracking sensor, an acoustical tracking sensor, or an optical tracking sensor such as a photogrammetry sensor, or active and passive IR markers.

Based on the signals received from the tracking sensor 110, the eye tracking sensor 118, and the position sensor 120, the processor 104 may determine a spatial relationship between the object 108, and the eye 122 of the operator and the imaging device 102. Scan information of the object 108 may be displayed at a location in the operator's line of sight. The image may be perceived by the operator 112 as an overlay to the object 108.

The imaging system 100 may also include additional tracking devices for tracking movement of the upper and/or lower jaw. Such tracking device(s) may provide additional information between the HMD 106 and the object 108. These tracking sensors also may utilize magnetic field tracking technology, active or passive Infrared tracking technology, acoustic tracking technology, or optical technology, photogrammetry technology, or any combination thereof.

Although embodiments of the invention are described in detail, it should be understood that various changes, substitutions and alterations can be made hereto without departing from the spirit and scope of the invention as described by the appended claims. An example of the intra-oral imaging system described above may include a three-dimensional imaging device transmitting modulated laser light from a light source at high frequency for the purpose of reducing coherence of the laser source and reducing speckle. The intra-oral imaging system may focus light onto an area of an object to image a portion of the object. The HMD may include a corrective lens on which the computer-generated image is projected or displayed, where the corrective lens corrects the vision of the operator. The HMD may include a monochromatic or a color display.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

1. An imaging system comprising:
a three dimensional (3D) imaging device configured to generate a dataset representative characteristics of at least a portion of a surface of an object;
a processor coupled to the 3D imaging device, the processor being configured to receive the dataset from the 3D imaging device and generate signals representative of a visual image of the surface of the object, the signals being based on the dataset as the dataset is received by the processor; and
a display configured to receive the signals representative of the visual image and to display the visual image in a field of view of an operator.

2. The imaging system of claim 1 where the three-dimensional imaging system comprises an intra-oral probe configured to capture a three dimensional image.

3. The imaging system of claim 1 where the display is coupled to a forward-most part of an operator's body and is configured to display the visual image substantially simultaneously as the dataset is generated.

4. The imaging system of claim 1 where the 3D imaging device comprises a tracking sensor configured to generate signals representative of a position of the 3D imaging device relative to an origin.

5. The imaging system of claim 4 where the tracking sensor is further configured to generate signals representative of an orientation of the intra-oral device relative to the origin.

6. The imaging system of claim 5 where the tracking sensor is one of a magnetic field sensor, an acoustical tracking sensor, an optical tracking sensor or any combination thereof.

7. The imaging system of claim 1 where the display comprises at least one position sensor configured to generate signals representative of a position of the display relative to an origin.

8. The imaging system of claim 7 where the at least one position sensor is further configured to generate signals representative of a position of the display relative to the origin.

9. The imaging system of claim 8 where the position sensor is one of a magnetic field sensor, an acoustical sensor, an optical sensor or any combination thereof.

10. A head-mounted display, comprising:
a see-through display configured to display a computer-generated image in a field of view of a user, the computer-generated image representing at least a surface characteristic of an object and being displayed in the field of view of the user having and an orientation and scale corresponding to a view of the object in the field of view of the user; and
a wearable processor configured to generate the computer-generated image based on a dataset generated by an intra-oral imaging system.

11. The head-mounted display of claim 10 further comprising a headband configured to position the see-through display in the field of view of the user.

12. The head-mounted display of claim 10 where the computer-generated image comprises a three-dimensional representation of at least a surface characteristic of the object.

13. The head-mounted display of claim 10 where the wearable processor is mounted with a headband.

14. The head-mounted display of claim 10 where the intra-oral imaging system comprises a three dimensional (3D) intra-oral imaging device configured to scan a surface of a dental item with light and generates a dataset representative of surface characteristics of scanned dental item in response to detecting a reflected light from the scanned surface.

15. The head-mounted display of claim 14 where the intra-oral imaging system further comprises at least one tracking sensor configured to generate signals representative of a position and an orientation of the intra-oral device relative to an origin and the head-mounted display comprises at least one position sensor configured to generate signals representative of a position of the head-mounted display relative to the origin, the wearable processor being configured to generate the computer-generated image in the field of view of the user based on the signals representative of a position of the intra-oral device and the electrical signals representative of a position of the head-mounted display.

16. The imaging system of claim 15 where the at least one tracking sensor is one of a magnetic field sensor, an acoustical sensor, an optical sensor or a combination thereof and the position sensor is one of a magnetic field sensor, an acoustical tracking sensor, an optical tracking sensor or any combination thereof.

17. A method of displaying a visual image:
projecting structured light towards a surface of an object;
detecting the structured light reflected from the surface of the object;
generating a dataset representative of the surface in response to the detecting of light reflected from the scanned surface of the object;
generating an image of the object based on the dataset;
displaying the image in a field of view of an operator on a see-through screen, the field of view of the operator including a view of the object, the image being displayed at a position and orientation in the field of view of the operator.

18. The method for displaying a visual image of claim 17, where the act of displaying the image further comprises:
detecting the position of the field of view of the operator;
detecting the position of the intra-oral imaging device; and
determining a shape and orientation of the image based on detecting the position of the field of view and detecting the position of the intra-oral imaging device.

19. The method for displaying a visual image of claim 17, further comprising:
determining an area of interest in the image; and
displaying the area of interest through the display.

20. The method for displaying a visual image of claim 17 where the image comprises a three-dimensional visual representation of at least a portion of the object.
